(19) United States
(12) Patent Application Publication
(10) Pub. No.: US 2007/0087059 A1
(43) Pub. Date: Apr. 19, 2007

(54) Bioactive delivery matrix compositions and methods

(76) Inventors: Frank Everaerts, Maastricht (NL); Marc Hendriks, Brussum (NL); Mirian Gillissen, Gulpen (NL)

Correspondence Address: MUETING, RAASCH & GEBHARDT, P.A., P.O. BOX 581415, MINNEAPOLIS, MN, 55458, US

(21) Appl. No.: 11/252,197

(22) Filed: Oct. 17, 2005

(51) Int. Cl. A61K 9/14

(52) U.S. Cl. 424/484; 424/488

(57) ABSTRACT

Bioactive delivery matrix compositions and methods of making and using such compositions.

### BACKGROUND

Various materials have been used to repair or regenerate bone or soft tissue that has been lost due to either trauma or disease. Typically, implantable bone repair materials provide a porous matrix (i.e., scaffolding) for the migration, proliferation, and subsequent differentiation of cells responsible for osteogenesis. While the compositions provided by this approach provide a stable structure for invasive bone growth, they do not promote bone cell proliferation or bone regeneration.

Subsequent approaches have used bone repair matrices containing bioactive proteins which when implanted into the bone defect provide not only a scaffolding for invasive bone ingrowth, but active induction of bone cell replication and differentiation. In general, these osteoinductive compositions include a matrix that provides the scaffolding for invasive growth of the bone and anchorage dependent cells and an osteoinductive protein source. The matrix may be selected from a variety of biocompatible materials including natural polymers, synthetic polymers, or inorganic materials such as a biodegradable porous ceramics. Two specific substances that have been found to induce the formation of new bone through the process of osteogenesis include demineralized bone particles or powder and bone morphogenetic proteins (BMPs).

While a wide variety of compositions have been used for tissue engineering, there still exists a need for improvements or enhancements, which would accelerate and enhance bone and soft tissue repair and regeneration thereby allowing for a faster recovery and a better result for a patient receiving the implant.

### SUMMARY

The present invention is directed to bioactive delivery matrix compositions and methods of making and using such compositions. In certain embodiments of the present invention, the compositions may include a crosslinking agent for subsequent crosslinking, for example. Alternatively, the compositions may be crosslinked prior to use. Such compositions can be used as implants for promoting bone growth.

In one embodiment, the present invention provides a bioactive delivery matrix composition that includes: a biocompatible polymer including thiol groups; a bioactive substance including proteins that promote bone formation; and a crosslinking agent for crosslinking at least a portion of the thiol groups. Preferably, the biocompatible polymer also includes other groups such as amine groups, carboxyl groups, hydroxyl groups, or combinations thereof.

The bioactive polymer is preferably naturally derived. For example, naturally derived polymers are selected from the group consisting of polysaccharides, proteins (of a wide variety of molecular weights), glycoaminoglycans, lipids and combinations thereof. Examples include, but are not limited to, collagen, alginates, chitosan, hyaluronic acid, celluloses, starches, fats, gelatin, and silk. Preferably, the biocompatible polymer is collagen wherein at least a portion of the amine groups, carboxyl groups, and/or hydroxyl groups have been replaced by thiol groups.

In certain embodiments, the bioactive substance is selected from the group consisting of demineralized bone matrix, bone marrow, artificial bone comprising hydroxyapatite and tri-calcium phosphate having proteins that promote bone formation associated therewith, and combinations thereof. Preferably, the bioactive substance is demineralized bone matrix.

In another embodiment, the present invention provides a bioactive delivery matrix composition that includes: a biocompatible polymer including crosslinked thiol groups (which form —S—S— bonds), and uncrosslinked groups comprising amine groups, carboxyl groups, and/or hydroxyl groups; and a bioactive substance including proteins that promote bone formation.

In another embodiment, the present invention provides a bioactive delivery matrix composition that includes demineralized bone matrix and collagen crosslinked through thiol groups.

The present invention also provides methods of making and using the compositions of the described herein.

In one embodiment, the present invention provides a method of preparing a bioactive delivery matrix composition, the method including: providing a biocompatible polymer including amine groups, carboxyl groups, and/or hydroxyl groups; replacing at least a portion of the amine groups, carboxyl groups, and/or hydroxyl groups with thiol groups; providing a bioactive substance comprising proteins that promote bone formation; mixing the biocompatible polymer with the bioactive substance; and crosslinking the thiol groups of the biocompatible polymer to form a crosslinked biocompatible polymer having disulfide bonds (—S—S— bonds).

In certain embodiments, the crosslinking occurs prior to mixing the biocompatible polymer with the bioactive substance. Alternatively, in certain embodiments, the crosslinking occurs after mixing the biocompatible polymer with the bioactive substance.

In certain embodiments, methods of the present invention can include freeze-drying the crosslinked polymer prior to mixing the biocompatible polymer with the bioactive substance. Alternatively, in certain embodiments, the crosslinking occurs after mixing the biocompatible polymer with the bioactive substance, and the method further includes freeze-drying the crosslinked polymer with the bioactive substance mixed therein.

The present invention also provides methods of delivering a bioactive substance to a subject by contacting the subject with a bioactive delivery matrix composition described herein. In such methods, crosslinking the biocompatible polymer can occur before or after contacting the subject with the composition.

The present invention also provides bioprosthetic devices that include a bioactive delivery matrix composition of the present invention.

The terms “comprises” and variations thereof do not have a limiting meaning where these terms appear in the description and claims.

As used herein, “a,” “an,” “the,” “at least one,” and “one or more” are used interchangeably. Thus, for example, a bioactive delivery matrix composition that comprises “a” bioactive substance can be interpreted to mean that the composition includes “one or more” bioactive substances.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is a bar graft showing the amount of converted amine groups using THF or dioxane-water as reaction solvent.

### DETAILED DESCRIPTION OF ILLUSTRATIVE EMBODIMENTS

The present invention is directed to bioactive delivery matrix compositions and methods of making and using such compositions. In a particularly preferred embodiment, the present invention provides a composition that includes demineralized bone matrix (DBM) or other bioactive substances that include proteins that promote bone formation in a carrier that includes collagen, other biocompatible polymers (e.g., naturally derived polymers such as chitosan and hyaluronic acid) or bone marrow. Such compositions can be used as implants that provide both osteoinductive and osteoconductive properties for the promotion of bone formation. Preferably, such compositions have an extended duration after implantation in a patient.

According to further embodiment of the invention, a chemical crosslinking method is provided. In certain embodiments of the present invention, the compositions may include a crosslinking agent for subsequent crosslinking. Alternatively, the compositions may be crosslinked prior to use. In a particularly preferred embodiment, during crosslinking, molecules of the biocompatible polymers (e.g., collagen) can be crosslinked together through thiol groups present on the bioactive polymer molecules; thereby forming disulfide bonds (—S—S— bonds). Also during crosslinking, biocompatible polymer molecules can be crosslinked to the bioactive substance (e.g., DBM) due to the presence of reactive surface groups on the bioactive substance. As a result, the present invention preferably provides an osteoinductive and osteoconductive matrix that lasts longer after implantation and that can still be turned over in vivo as bone is formed. This method also allows control of the amount of bioactive substance added to the matrix and optimization of the material handling characteristics of the resulting composition.

In a preferred embodiment, the present invention provides a bioactive delivery matrix composition that includes: a biocompatible polymer having thiol groups; a bioactive substance that includes proteins that promote bone formation; and a crosslinking agent for crosslinking at least a portion of the thiol groups (thereby forming —S—S— bonds). In a particularly preferred embodiment, the present invention provides a bioactive delivery matrix composition that includes: a biocompatible polymer having crosslinked thiol groups, and uncrosslinked groups including amine groups, carboxyl groups, and/or hydroxyl groups; and a bioactive substance that includes proteins that promote bone formation. The crosslinked biocompatible polymer forms the matrix in which the bioactive substance is incorporated.

Thus, in preferred embodiments, the biocompatible polymer also includes amine groups, carboxyl groups, and/or hydroxyl groups. Preferably, the biocompatible polymer is a naturally derived polymer. For example, naturally derived polymers are selected from the group consisting of polysaccharides, proteins (having a wide variety of molecular weights), glycoaminoglycans, lipids and combinations thereof. Examples include, but are not limited to, collagen, alginates, chitosan, hyaluronic acid, celluloses, starches, fats, gelatin, and silk. The biocompatible polymer may be patient-derived or prepared through recombinant technology. The amine groups, carboxyl groups, and/or hydroxyl groups may be converted to thiol groups before the material is implanted.

In particularly preferred embodiments, the biocompatible polymer is collagen. The collagen source can be allogeneic or xenogeneic relative to the mammal receiving the implant. The source of the collagen may be from human or animal sources, or could be in a recombinant form expressed from a cell line or bacteria. The recombinant collagen may be from yeast or from any prokaryotic cell. The collagen may be extracted from tissue by any known method. The collagen protein can be any type of collagen, but particularly preferred is type I collagen.

Amine groups (i.e., amino groups), carboxyl groups, and/or hydroxyl groups can be converted to thiol groups using a variety of techniques. For example, amine groups can be reacted with S-acetylmercaptosuccinic anhydride and the resultant —S—C(O)—CH₃ groups deprotected with hydroxylamine to yield free thiol groups (—SH groups). In certain embodiments, carboxyl groups can be converted to amine groups before the amine groups are replaced by thiol groups. This can be done, for example, by reaction of the carboxyl groups with a diamine, such as ethylenediamine in presence of 1-ethyl-3-(3-dimethylaminopropyl)-carbodiimide (EDC). Hydroxyl groups can also be converted to thiol groups directly, for example, by activation of the hydroxyl groups with toluenesulfonyl chloride and subsequent transesterification in thioacetate solution. Hydrolysis of the thioester can be done with sodium methanoate (Ebert et al., In Protein Cross-linking, Fridman, Ed., Plenum Press, New York, 1977). These reactions are typically carried out in organic solvents, which along with the various conditions of temperature, pH, and time, can be readily determined by one of skill in the art without undue experimentation in such methods of forming thiol groups.

In another example, the modification of amine to thiol groups can be carried out as described in U.S. Pat. No. 5,412,076; however, this method is complex and difficult to carry out. Various conditions of temperature, pH, and time, can be used as readily determined by one of skill in the art without undue experimentation.

A method of forming —SH groups on a biocompatible polymer without any organic solvents is described in Example 2. Briefly, in this method, at least a portion of the amine groups of a biocompatible polymer (e.g., collagen) are reacted with a blocking agent, such as an aldehyde (e.g., propional), as described in U.S. Pat. No. 6,166,184 (Hendriks et al.) and at least a portion of the carboxyl and/or hydroxyl groups are reacted with cystamine to form thiol groups. The formation of the thiol groups is preferably enhanced by the presence of an activating agent (e.g., a carbodiimide), examples of which are described in U.S. Pat. No. 6,166,184 (Hendriks et al.).

In preferred embodiments, the bioactive substance is selected from the group consisting of bone marrow, demineralized bone matrix, artificial bone having hydroxyapatite and tri-calcium phosphate having proteins that promote bone formation associated therewith, and combinations thereof. In this context, “associated therewith” refers to proteins that are covalently or otherwise attached or absorbed or ‘locked’ (imbibed) in the artificial bone. In this context, “proteins that promote bone formation” include, for example, BMP's, VEGF and family of heparin binding growth factors (Street J, et al., Proc. Natl. Acad. Sci. USA, 99: 9656-61, 2002).

The bioactive substance is preferably a natural component, which thus allows for cellular attachment and migration and can be remodeled by the cells present in the defect site. The bioactive substance may be patient-derived (directly) before the material is implanted.

In particularly preferred embodiments, the bioactive substance is demineralized bone matrix (DBM). Thus, in one embodiment, the present invention provides an implant that includes demineralized bone matrix and collagen crosslinked through thiol groups.

The bioactive substance (e.g., DBM) can be in the form of particles of any size or shape (e.g., blocks or strips). For example, particles having an average diameter of up to 5 millimeters (mm) can be used according to one embodiment of the invention. In some embodiments, the particles have an average diameter of no more than 4 mm, and in some embodiments, no more than 850 micrometers (μm). According to a further embodiment of the invention, particles having an average diameter of at least 2 mm can be used. According to another embodiment of the invention, the particles having an average diameter of at least 53 μm can be used. Larger or smaller particles can also be used, however, depending on the desired properties of the composition.

Compositions according to an embodiment of the invention can include any amount of bioactive substance (e.g., DBM). The amount of bioactive substance can be varied to achieve desired properties in the composition. According to one embodiment of the invention, the composition can include preferably at least 2 weight percent (wt-%), more preferably at least 25 wt-%, and even more preferably at least 50 wt-%, of the bioactive substance based on the combined weight of the bioactive substance and the biocompatible polymer. According to a further embodiment of the invention, the composition can include preferably no more than 95 wt-%, more preferably no more than 85 wt-%, and even more preferably no more than 75 wt-%, of the bioactive substance based on the combined weight of the bioactive substance and the biocompatible polymer.

As discussed above, compositions of the present invention may include a crosslinking agent. Preferred crosslinking agents are oxidizing agents. Suitable “oxidizing agents” are those molecules that, in combination with other molecules in the solution, provide the energy to form localized reduction-oxidation reactions that result in a transfer of electrons. No external energy source is required to generate these electrons, although, suitable crosslinking agents may be activated by an activating agent, such as water, for example.

Examples of suitable oxidizing agents include, for example, a peroxide, iodine, ferric sulfate, a mixture of copper chloride and hydrogen peroxide, or a mixture of ascorbate and ferrous chloride. The oxidizing agents generally have a simple structure and can be simple salts. These oxidizing agents can therefore be readily removed from the tissue when the crosslinking reactions are complete.

Without meaning to be bound by any theory, it is thought that an oxygen radical is an intermediate in the oxidation reaction. This oxygen radical, also called an oxygen singlet, is believed to be produced by a coupled oxidation-reduction reaction that consumes the oxidizing agent and cleaves dissolved oxygen molecules by electron transfer. Therefore, while the actual oxidizing species may be an intermediate that is formed in solution, as used herein the term “oxidizing agent” is meant to include those compounds that are precursors to the actual oxidizing agent, or catalysts promoting the formation of intermediate radicals that are the actual oxidizing agent, or compounds that donate electrons or hydrogen atoms, or any other compounds that may participate in the oxidation reaction that results in a cross-linked product. These oxidizing reagents provide the energy to drive the reduction/oxidation reaction that directly or indirectly results in oxidation of the proteinaceous material. No external energy, in the form of light, or heat, or electrical current, need be added to the solution.

The type and amount of crosslinking agents are selected to be sufficient to crosslink preferably at least 10%, more preferably at least 20%, even more preferably at least 30%, even more preferably at least 40%, even more preferably at least 50%, even more preferably at least 60%, even more preferably at least 70%, even more preferably at least 80%, even more preferably at least 90%, and even more preferably at least 100%, of the thiol groups. Such crosslinking results in the formation of disulfide bonds.

The chemical crosslinking allows the amount of DBM or other bioactive substance added to the matrix and the material handling characteristics to be optimized without significantly affecting the osteoinductive and osteoconductive capacity of the DBM or other bioactive substance.

In a preferred embodiment, the present invention provides a method of preparing a bioactive delivery matrix composition that includes: providing a biocompatible polymer having amine groups, carboxyl groups, and/or hydroxyl groups; replacing at least a portion of the active groups with thiol groups; providing a bioactive substance comprising proteins that promote bone formation; mixing the biocompatible polymer with the bioactive substance; and crosslinking the thiol groups of the biocompatible polymer to form a crosslinked biocompatible polymer having disulfide bonds.

In certain methods, the crosslinking can occur prior to mixing the biocompatible polymer with the bioactive substance. Alternatively, it can occur after mixing the biocompatible polymer with the bioactive substance.

In certain embodiments, methods of the present invention can include freeze-drying the crosslinked polymer prior to mixing the biocompatible polymer with the bioactive substance. Alternatively, crosslinking can occur after mixing the biocompatible polymer with the bioactive substance, and methods of the present invention can further include freeze-drying the crosslinked polymer with the bioactive substance mixed therein.

Crosslinked or uncrosslinked compositions can be delivered to a subject. If the composition is uncrosslinked, crosslinking of the biocompatible polymer can occur after contacting the subject with the composition.

Crosslinking of thiol groups can occur using a variety of techniques. In particular, the thiol groups are preferably crosslinked with an oxidizing agent, such as a peroxide or iodine. Various conditions of temperature, pH, and time can be used as readily determined by one of skill in the art without undue experimentation.

In a typical crosslinking reaction, the uncrosslinked material is immersed in an oxidizing solution for a specified period of time at a specified temperature. Both the temperature and the time can be widely varying and are of limited importance. Immersion times may range from minutes to hours or even days. To a point, the longer the immersion time the greater the extent of crosslinking. The temperature of the solution is also not important. As with most catalytic or kinetic type reactions, the reaction rate increases with temperature; however, if the solution gets too warm the proteins will become denatured by the heat. In addition, the solubility, and therefore the availability, of dissolved oxygen declines with increased temperature. It is possible to oxidize proteinaceous tissue from about the freezing point of the solution used to 40° C. Higher temperatures, from 20° C. to about 40° C. are preferred.

Compositions of the present invention may be in one part (e.g., a crosslinked composition), or in two or more parts. For example, the crosslinking agent can be in a separate container. Alternatively, the biocompatible polymer, bioactive substance, and crosslinking agent can all be in separate containers.

A bioactive delivery matrix composition can be in the form of an implant or associated with a bioprosthetic device. Such devices include, for example, a cage material of a metal or biodegradable material (e.g., of the type used in replacing vertebral discs) or a sheet of a biodegradable material. Such devices are typically filled with bioactive delivery matrix compositions of the present invention.

The bioactive delivery matrix composition can be in a wide variety of shapes suitable, for example, for implantation. In particular, the crosslinking allows for the production of a bioactive delivery matrix composition that can maintain its shape, for example, when hydrated. Furthermore, in certain embodiments, the bioactive delivery matrix composition can regain its height following compression, for example, when hydrated. The bioactive delivery matrix composition according to one embodiment of the invention can be in the form of a block, a gel, a powder, a putty, a paste, a sponge, a membrane, a fiber-like structure, a fleece, particles, fibers, or a viscous solution, for example. It can be cut into various shapes. It can be rolled to fit into a variety of configurations.

Preferably, the bioactive delivery matrix composition is in the form of porous or semi-porous scaffolding that can provide an osteoconductive and osteoinductive matrix for bone in-growth. Any known method of forming porous or semi-porous scaffolding can be used. For example, a bioactive substance (e.g., DBM) and a biocompatible polymer (e.g., collagen) in the form of slurry (e.g., an aqueous slurry) can be cast into the cavity of a mold having the desired shape and freeze dried to form the scaffolding. After the dried scaffolding is removed from the mold, the crosslinking agent can then be infiltrated into the pores of the composition and allowed to react with the biocompatible polymer and the bioactive substance to form the crosslinks.

If the bioactive delivery matrix composition is in the form of sponges, they can be in the shape of cubes or rectangular solids with dimensions of 2 millimeters (mm) to 10 mm, for example. These can be packed into a defect site for bone or soft tissue repair. If desired, the sponges can be ground to a finer size and combined with saline or another diluent (e.g., blood) to create a paste material. This paste can be injected or packed into a wound site for bone or soft tissue repair.

The composition can remain intact within the implant site for a 6- to 10-week time frame, for example. This time frame, however, depends on implantation site and patient-to-patient variability.

Compositions of the present invention can also include one or more growth factors. The one or more growth factors can be present within or on the matrix. For example, cytokines or prostaglandins may be present within or on the porous or semi-porous collagen matrix or within or on the DBM particles. The growth factor may be of natural origin or recombinantly or otherwise produced using conventional methods. Such growth factors are also commercially available. Combinations of two or more growth factors may be applied to the compositions to further enhance the osteoinductive and osteoconductive properties or other biologic activity of the implants.

Examples of growth factors that may be used, include, but are not limited to: transforming growth factor-beta (TGF-beta), such as TGF-beta-1, TGF-beta-2, and TGF-beta-3; transforming growth factor-alpha (TGF-alpha); epidermal growth factor (EGF); insulin like growth factor-I or II; interleukin-I (IL-I); interferon; tumor necrosis factor; fibroblast growth factor (FGF); platelet derived growth factor (PDGF); BMP, VEGF, nerve growth factor (NGF); and other molecules that exhibit growth factor or growth factor-like effects. According to one embodiment of the invention, the growth factor can be a soluble growth factor.

The growth factor may be incorporated into the biocompatible polymer (e.g., collagen) prior to or after incorporating the bioactive substance (e.g., DBM) therein, prior to or after crosslinking the biocompatible polymer. For example, the growth factor(s) may be adsorbed onto the crosslinked biocompatible polymer matrix in an aqueous or non-aqueous solution. Alternatively, a solution comprising the growth factor may be infiltrated into the uncrosslinked matrix. According to a further embodiment, a solution comprising the growth factor may be infiltrated into the crosslinked matrix using vacuum infiltration. The growth factor(s) can also be provided in a dry state prior to reconstitution and administration onto or into the biocompatible polymer (with or without the bioactive substance therein). The growth factor(s) present on or within the matrix may reside, for example, within the void volume of a porous or semi-porous matrix. Growth factor(s) contained within a controlled release carrier may also be used.

Cells, plasticizers, and calcium- or phosphate-containing compounds can also be added to compositions according to an embodiment of the invention. Examples of suitable cells include osteoblasts, osteoclasts, progenitor cells, and stem cells. Examples of suitable plasticizers include natural waxes made of a mixture of alcohols, fatty acids and esters (such as bees-wax), and the like. Examples of calcium- or phosphate-containing compounds include ceramics made of for example hydroxyapatite and/or tricalciumphosphate.

A bioactive delivery matrix composition of the present invention can be used alone or combined with allograft or autograft tissue for bone or soft tissue repair. Examples of such tissue include minced bone and marrow.

Following is a description of non-limiting examples of reaction methods that can be used to form crosslinked collagen/DBM compositions.

### EXAMPLES

Objects and advantages of this invention are further illustrated by the following examples, but the particular materials and amounts thereof recited in these examples, as well as other conditions and details, should not be construed to unduly limit this invention.

### Example 1

The aim of experimental work performed and described in Example 1 was to demonstrate that collagen —COOH end groups could be modified to —SH-containing moieties.

Materials and Methods

A sample of 0.1 gram (g) collagen powder (Kensey Nash, fibrous porcine collagen, Germany) or collagen discs (DSC, Coletica bovine collagen, France) were suspended either in 5 milliliters (mL) tetrahydrofuran (THF, Aldrich, the Netherlands), or in 4.5 mL dioxane (Merck, the Netherlands) with the addition of 0.5 mL deionized water (DiW), in capped vials. A sample of 250 milligrams (mg) of S-acetylmercapto succinic anhydride (SASA, Aldrich, the Netherlands) was added to the suspension. The reaction was continued for 2 and 24 hours (h).

The suspensions were filtered using a Buchner filter and washed with THF followed by ethanol. The residues were dried over night under vacuum at room temperature.

Per dried sample, 0.25 g of hydroxylamine (Aldrich, the Netherlands) was dissolved in a mixture of ethanol/Phosphate Buffer Saline (PBS, 4.5 mL/0.5 mL, Aldrich, the Netherlands). The samples were incubated with this mixture for either 2 or 24 hours. After filtration the residues were rinsed with a mixture of 5% acetic acid and 95% ethanol. The samples were dried over night under vacuum, at room temperature.

The samples were either rehydrated in PBS (2 mL) or ethanol (2 mL) containing 1% acetic acid.

Crosslinking was initiated by the addition of either 30 wt-% peroxide (0.2 mL, Aldrich, the Netherlands), or a saturated solution of iodine (0.2 mL Fluka, the Netherlands) in methanol.

Results

In order to test the efficacy of the modification reaction of the amine groups, a colorimetric assay using 2,4,6-trinitrobenzene sulfonic acid (TNBS) was used (Everaerts F, et al., Biomaterials 25: 5523-5530, 2004).

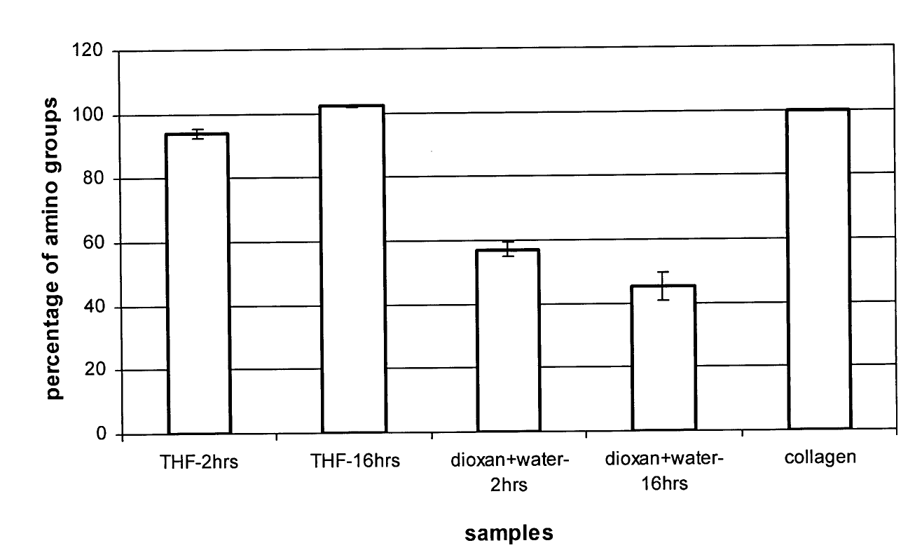

The results in FIG. 1 show that a significant amount of amine groups could be modified when using dioxane, while with THF only a small amount of amine groups could be modified.

After the crosslinking reaction was completed using iodine or peroxide, a stable gel was obtained at 20° C. In this example, a stable gel was obtained at 20° C., however, not at 37° C. It is believed that more —S—S— crosslinks will give a gel that is stable at 37° C.

### Example 2

The aim of experimental work performed and described in Example 2 was to demonstrate that with an alternative method not requiring any organic solvent, collagen —COOH end groups could be modified to —SH-containing moieties.

Materials and Methods

Bovine collagen discs (6 mm, Coletica, France) were rehydrated in 2-(morpholino) ethane sulfonic acid buffer (MES; 0.05 Molar (M), pH 6.5). After 1 hour (h) propional (to a total concentration of 0.5 M, Aldrich, USA) and NaCNBH₃ (50 mM, Aldrich, USA) was added. The blocking reaction was allowed to continue for 4 h followed by rinsing in sterile saline (for 20 h with solution changes at 1 h, 2 h, 8 h, and 20 h). The collagen discs were introduced in a MES buffer (0.25 Molar (M), pH 5.0) containing cystamine at a concentration of 0.39 M or 0.039 M (Sigma Aldrich, Netherlands). Then an equal amount of a concentrated solution of N-hydroxysuccinimide (NHS, 0.45 M, Sigma Aldrich, USA) and an equal amount of a concentrated solution of N′-(3-dimethylaminopropyl)-N-ethylcarbodiimide (EDC, 0.9 M, Aldrich, USA) both in MES buffer (0.25 M, pH 5.0) were added. The reaction was allowed to proceed for 2 h or 7 h and the material was subsequently rinsed in saline (for 24 h with solution changes at 1 h, 2 h, 8 h and 20 h). The material was freeze dried afterwards.

The amount of —SH groups was determined using a technique using dithionitrobenzoic acid (DTNB) as described in U.S. Pat. No. 5,412,076. The conversion rate was calculated based on the assumption that the used native collagen contains 120-COOH groups per 1000 amino acid residues. The conversion rate is depicted in the next table:

| Sample | Cystamine | Reaction | Conversion to |
|---|---|---|---|
| Number | concentration (M) | time (h) | SH (%) |
| A | 0.39 | 7 | 24 |
| B | 0.039 | 7 | 11 |
| C | 0.39 | 2 | 28 |

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A bioactive delivery matrix composition comprising:
a biocompatible polymer comprising thiol groups;
a bioactive substance comprising proteins that promote bone formation; and
a crosslinking agent for crosslinking at least a portion of the thiol groups.

2. The bioactive delivery matrix of claim 1 wherein the biocompatible polymer further comprises amine groups, carboxyl groups, and/or hydroxyl groups.

3. The bioactive delivery matrix of claim 1 which is in the form of a block, a gel, a powder, a putty, a paste, a sponge, a membrane, a fiber-like structure, a fleece, particles, fibers, or a viscous solution.

4. The bioactive delivery matrix of claim 1 which is in two or more parts.

5. The bioactive delivery matrix of claim 1 wherein the biocompatible polymer is a naturally derived polymer.

6. The bioactive delivery matrix of claim 5 wherein the naturally derived polymer is selected from the group consisting of polysaccharides, proteins, glycoaminoglycans, and combinations thereof.

7. The bioactive delivery matrix of claim 5 wherein the naturally derived polymer is selected from the group consisting of collagen, alginates, chitosan, hyaluronic acid, celluloses, starches, proteins, fats, gelatin, and silk.

8. The bioactive delivery matrix of claim 7 wherein the biocompatible polymer is collagen.

9. The bioactive delivery matrix of claim 1 wherein the bioactive substance is selected from the group consisting of demineralized bone matrix, bone marrow, artificial bone comprising hydroxyapatite and tri-calcium phosphate having proteins that promote bone formation associated therewith, and combinations thereof.

10. The bioactive delivery matrix of claim 9 wherein the bioactive substance is demineralized bone matrix.

11. The bioactive delivery matrix of claim 1 wherein the crosslinking agent is an oxidizing agent.

12. The bioactive delivery matrix of claim 11 wherein the oxidizing agent comprises a peroxide, iodine, ferric sulfate, a mixture of copper chloride and hydrogen peroxide, or a mixture of ascorbate and ferrous chloride.

13. The bioactive delivery matrix of claim 1 wherein the crosslinking agent is sufficient to crosslink at least 10% of the thiol groups.

14. A bioactive delivery matrix composition comprising:
a biocompatible polymer comprising crosslinked thiol groups, and uncrosslinked groups comprising amine groups, carboxyl groups, and/or hydroxyl groups; and a bioactive substance comprising proteins that promote bone formation.

15. The bioactive delivery matrix of claim 14 wherein the bioactive substance is selected from the group consisting of bone marrow, demineralized bone matrix, artificial bone comprising hydroxyapatite and tri-calcium phosphate having proteins that promote bone formation associated therewith, and combinations thereof.

16. The bioactive delivery matrix of claim 15 wherein the bioactive substance is demineralized bone matrix.

17. The bioactive delivery matrix of claim 14 wherein the biocompatible polymer is a naturally derived polymer.

18. The bioactive delivery matrix of claim 17 wherein the naturally derived polymer is selected from the group consisting of polysaccharides, proteins, lipids, glycoaminoglycans, and combinations thereof.

19. The bioactive delivery matrix of claim 17 wherein the naturally derived polymer is selected from the group consisting of collagen, alginates, chitosan, hyaluronic acid, celluloses, starches, fats, gelatin, and silk.

20. The bioactive delivery matrix of claim 19 wherein the biocompatible polymer is collagen.

21. An implant comprising demineralized bone matrix and collagen crosslinked through thiol groups.

22. A method of preparing a bioactive delivery matrix composition, the method comprising:
providing a biocompatible polymer comprising amine groups, carboxyl groups, and/or hydroxyl groups;
replacing at least a portion of the amine groups, carboxyl groups, and/or hydroxyl groups with thiol groups;
providing a bioactive substance comprising proteins that promote bone formation;
mixing the biocompatible polymer with the bioactive substance; and
crosslinking the thiol groups of the biocompatible polymer to form a crosslinked biocompatible polymer comprising disulfide bonds.

23. The method of claim 22 wherein the crosslinking occurs prior to mixing the biocompatible polymer with the bioactive substance.

24. The method of claim 22 further comprising freeze-drying the crosslinked polymer prior to mixing the biocompatible polymer with the bioactive substance.

25. The method of claim 22 wherein the crosslinking occurs after mixing the biocompatible polymer with the bioactive substance, and the method further comprises freeze-drying the crosslinked polymer with the bioactive substance mixed therein.

26. The method of claim 22 wherein the biocompatible polymer with the bioactive substance mixed therein is formed into a block, a gel, a powder, a putty, a paste, a sponge, a membrane, a fiber-like structure, a fleece, particles, fibers, or a viscous solution.

27. The method of claim 22 wherein the biocompatible polymer comprises amine groups and replacing at least a portion of the amine groups with thiol groups comprises:
reacting the amine groups with S-acetylmercaptosuccinic anhydride to form —S—C(O)—CH₃ groups; and
reacting the —S—C(O)—CH₃ groups with for example hydroxylamine to yield free thiol groups.

28. The method of claim 27 wherein the biocompatible polymer comprises carboxyl groups and the method comprises converting the carboxyl groups to amine groups prior to reacting the amine groups with S-acetylmercaptosuccinic anhydride.

29. The method of claim 22 wherein replacing at least a portion of the amine groups, carboxyl groups, and/or hydroxyl groups with thiol groups comprises:
reacting at least a portion of the amine groups with a blocking agent; and
reacting at least a portion of the carboxyl and/or hydroxyl groups with cystamine to form thiol groups.

30. A method of delivering a bioactive substance to a subject, the method comprising contacting the subject with the bioactive delivery matrix composition of claim 1, and crosslinking the biocompatible polymer before or after contacting the subject with the composition.

31. A method of delivering a bioactive substance to a subject, the method comprising contacting the subject with the bioactive delivery matrix composition of claim 14.

32. A method of delivering a bioactive substance to a subject, the method comprising contacting the subject with the implant of claim 21.

33. A bioprosthetic device comprising the bioactive delivery matrix composition of claim 1.

34. A bioprosthetic device comprising the bioactive delivery matrix composition of claim 14.

35. A bioprosthetic device comprising the implant of claim 21.
